(19) United States
(12) Patent Application Publication
(10) Pub. No.: US 2005/0096507 A1
(43) Pub. Date: May 5, 2005

(54) Adjustable length cannula

(76) Inventor: Michael Ugene Prosek, Jacksonville, FL (US)

Correspondence Address: MICHAEL E. PROSEK, 4766 MARSH HAMMOCK DRIVE EAST, JACKSONVILLE, FL, 32224, US

(21) Appl. No.: 10/696,833

(22) Filed: Oct. 30, 2003

(51) Int. Cl. A61B 1/32

(52) U.S. Cl. 600/204; 606/1

(57) ABSTRACT

The Adjustable Length Cannula surgical instrument assembly may be provided with a valve seal assembly where the upper portion of the valve seal is rigidly mounted to the upper head end of the adjustable cannula body assembly. The lower portion of the adjustable cannula body is of two or more components that includes a gas seal that telescopes to multiple desired lengths by telescoping and securing in position with a short twist motion with a detent locking detail.

### CROSS-REFERENCE TO RELATED APPLICATIONS

U.S. Pat. Nos. 5,957,888 and 5,746,720 use common screw threads to change the length of the instruments, this is time consuming and perceived as difficult and complicated by the operating room surgical staff. The present invention uses a short rotational twist to unlock and lock and a push or pull telescopic action to set the device at the desired lengths.

### BACKGROUND OF THE INVENTION

The present invention relates broadly to medical devices used during surgical procedures and more particularly to surgical procedures that require cannulas of multiple lengths due to patient abdominal body cavity wall thickness or tissue thickness.

### BRIEF SUMMARY OF THE INVENTION

The present invention is comprised of two plastic molded resin or metal constructed components and can be of single use disposable or multi-use re-usable. It is inexpensive to manufacture and its ease of use will reduce training time required. Furthermore it will reduce stocking needs of the hospitals as compared to the current fixed length instrument counterparts.

Typically, in current devices the length of the cannula is a conduit allowing body cavity access through the body cavity abdominal wall for laparoscopic surgery is generally fixed at a specific length. These fixed length cannulas require the hospital to inventory cannulas of all commonly used lengths to meet the patient body abdominal body wall thickness or tissue thickness. The present invention allows the surgeon or operating room surgical staff to set the desired length with the one cannula relieving the hospital of excess inventory requirements.

### BRIEF DESCRIPTION OF THE DRAWINGS

FIG. 1 Illustrates an outside view of the complete device, ready for use set at a mid length configuration.

FIG. 2 Illustrates an outside view of the complete device, ready for use set at a maximum length configuration.

FIG. 2 Illustrates an outside view of the complete device, ready for use set at a mid length configuration.

FIG. 4 Illustrates an outside view of the complete device, ready for use set at a shortest length configuration.

FIG. 5 Illustrates an exploded view of the device with reference numerals.

FIG. 6 Illustrates an outside view of the complete device, ready for use set at a mid length configuration with reference numerals.

FIG. 7 Illustrates a full length section view with reference numerals.

FIG. 8 Illustrates an assembled view with a mid sectional area showing the gas sealing detail.

FIG. 9 Illustrates section “B-B” showing the detail of the twist-lock feature.

FIG. 10 Illustrates an exploded view with two sectional areas defined.

FIG. 11 Illustrates section “A-A”.

FIG. 12 Illustrates a 90 degree rotated view of the twist-lock lug(28) detail.

### REFERENCE NUMERALS

1. Cannula Head.

2. Inner Body Tube.

3. Twist-Lock Receiver.

4. Gas Seal.

5. Bevel.

6. Outer Body Tube fascia anchoring threads.

7. Conical Taper.

8. Slots.

9. Outer Body Tube.

10. Trough.

11. Gas Seal Inner Bevel.

12. Expanding Inner Taper.

13. Gas Seal Bore.

14. Instrument Bore.

15. Section View Showing Gas Seal Area.

16. Lug Stop/Lock Wall.

17. Lug Stop Pocket.

18. Lug Detent.

19. Lug Detent Ramp.

20. (Omitted)

21. Lug Guide Wall/Stop.

22. Lug Section View.

23. Lug.

24. Lug Lead-In Taper.

25. Lug Entrance.

### DETAILED DESCRIPTION OF THE INVENTION

1. An outer body tube (9) that embodies a smooth tubular interior surface with a protruding alignment/locking lug (23). The bevel (5) provides for a smooth transition of outer body tube (9) and inner body tube (2) diameters reducing tissue trauma upon device removal from the body cavity abdominal wall. Generally made from polypropylene, polycarbonate, ABS or other plastic resins for disposable one-time use or Radel, Ultem or other autoclaveable plastic resins including metals allowing for user autoclaving sterilization methods.

2. The inner body tube (2) slip fits within the outer body tube (9) and embodies a smooth, thin, flexible flared edge lip gas seal (4) this sealing lip is in light contact sufficient to provide a gas seal, creates and maintains the gas seal while allowing free extension and retraction of the two members via a lengthwise trough (10). Generally made from polypropylene, polycarbonate, ABS or other plastic resins for disposable one-time use or Radel, Ultem or other autoclaveable plastic resins including metals allowing for user autoclaving sterilization methods.

3. Multiple slots or twist-lock receivers (3) intersecting perpendicular with the trough (10) include a sufficient funnel like lug entrance (25) for the lug (23) providing easy location and engagement for setting the desired length.

4. A lug detent (18) is a smooth radius cam like detail for receiving and engaging the protruding alignment locking lug (23) located inside the bore of the outer body, provides for the short twist motion to engage the lug (23) detail by first contacting the lug detent ramp (19) that provides for the means to expand the outer body tube (9) and deflect the inner body tube (2) that in combination allows the lug (23) to traverse the apogee of the lug detent (18) and then falling into the lug pocket stop (17) thereby holding securely in position the two body members at the selected length.

5. The gas seal (4) also comprises a conical taper located in the inside smooth diameter of the inner body tube (2) this conical taper creates the thin flared edged lip of the gas seal (4) also provides a smooth transition between the inner body tube (2) and the inside smooth bore of the outer body tube (9) thereby reducing the possibility of the surgical instruments used therethrough of catching or snagging onto.

6. A conical taper (7) eases the tissue dilation force required to insert the cannula assembly through the abdominal body cavity wall.

7. Eight multiple slots (more or less) about and through the conical taper (7) allow flexing for the removal of the molding core pin enabling this component to be injection molded.

8. An inner taper (12) is the area that allows for the transition of the inside diameter of the outer body tube (9) to match the inner body tube (2) thereby reducing undesirable dimensional gapping of the surgical instruments outside diameters used therethrough and the inside diameter of the inner body tube (2).

9. A lug twist-lock receiver (3) detail consists of a lug/stop wall (16) to abut the lug (23) firmly, limiting and indicating the selected position is secure. The lug/stop pocket (17) allows sufficient area to contain the lug (23) after it falls from the apogee of lug detent (18) detail.

10. A trough (10) provides the linear guidance and telescopic movement for the lug (23) as the device is telescoped to the users desired length. The lug guide wall/stop provides for a linear smooth surface for the lug (23) when slight pressure is applied in a counterclockwise direction during the telescoping of the outer body tube (9). Perpendicular at various positions are the twist-lock receivers (3) for receiving the lug (23). The twist-lock receivers (3) have a generous, lug entrance (25) tapered or radius entry for the lug (23) allowing the surgeon or assistant ease of locating and engagement to the desired length position.

11. A lug lead-in taper (24) is provided for device assembly of the outer body tube (9) onto the inner body tube (2).

The Adjustable Length Cannula is manufactured and shipped assembled ready for use.

The surgeon or operating room surgical nurse or surgical assistant can adjust the length of the device by grasping the outer body tube (9) with the distal end facing away from the user using one (left) hand and grasping the cannula head (1) with the other (right) hand. By rotating the cannula head (1) counterclockwise from the outer tube (2) until the lug (23) abuts the lug guide wall/stop (21) of the trough (10). The device is now able to be telescoped to the desired length suitable to the patients abdominal body cavity wall or tissue thickness. Upon selecting the suitable length position, as predetermined by the twist-lock receiver (3) locations, the user while still grasping the cannula head (1) rotates clockwise to engage the lug (23) into the twist-lock receiver (3) slight pressure is needed to pass the lug (23) over the detent (18) until the lug (23) drops into the lug stop pocket (17) and abuts with a felt click or vibration the lug stop/lock wall (16). The device length is now secured and ready for use.

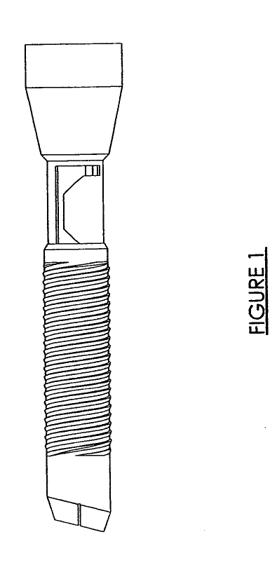

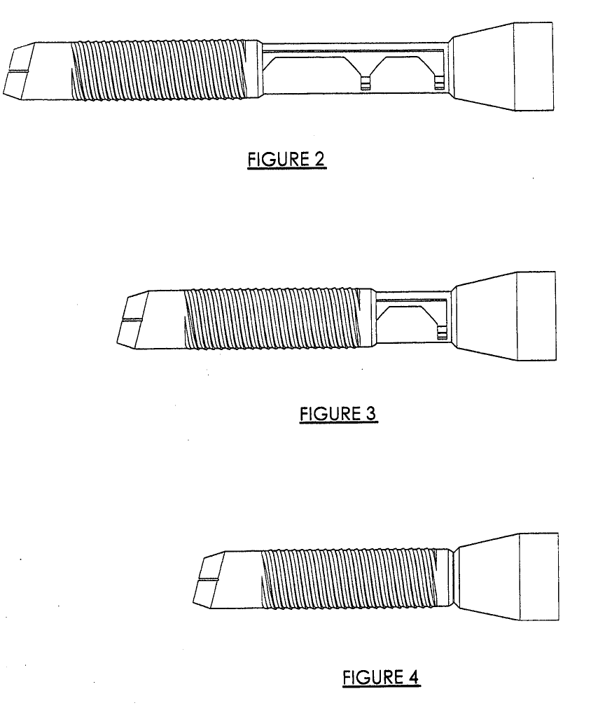

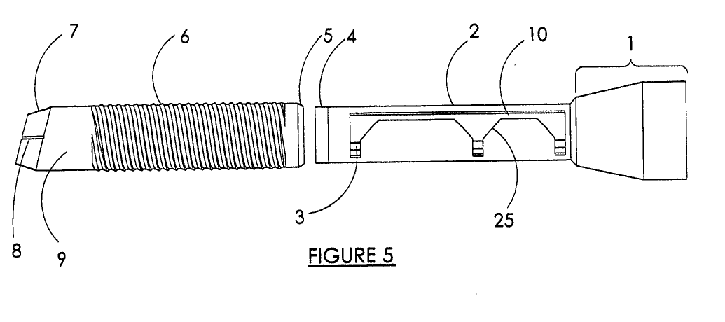

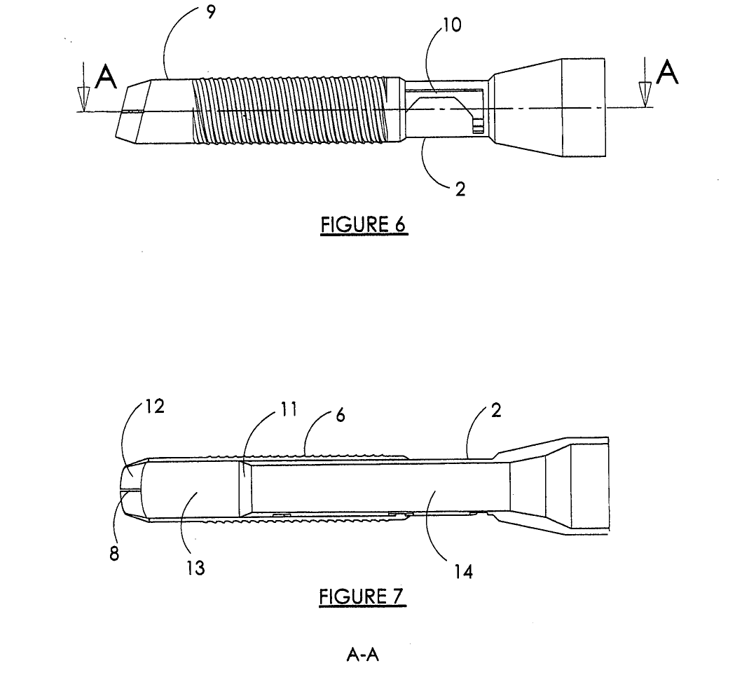

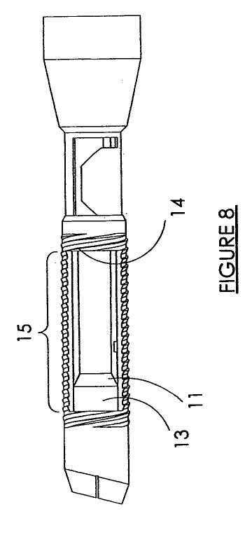

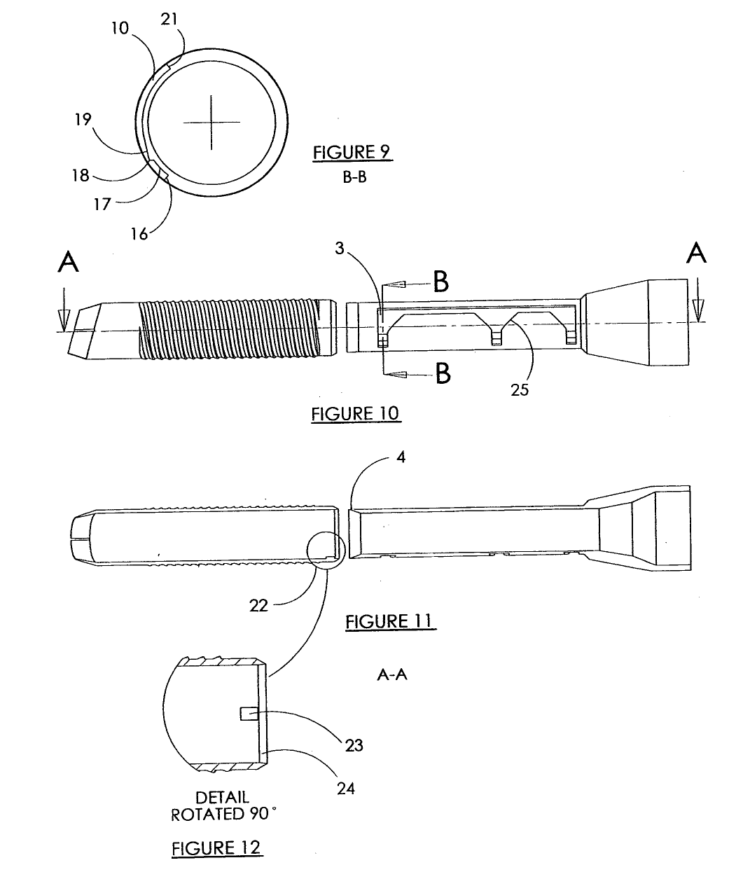

1. I claim the present invention, is a two component, Adjustable Length Cannula (FIG. 1) surgical instrument assembly for use in laparoscopic surgery as a conduit through the abdominal body cavity that allows the insertion of various surgical instruments therethrough.

2. I claim the present inventions, components are manufactured of a plastic resin injection molded or of metal components.

3. I claim the present invention, device can be manufactured as a single use disposable instrument.

4. I claim the present invention, can be manufactured as a multi-use instrument made from materials that allow autoclaving by the end user.

5. I claim the present invention, is comprised of a outer body tube (9) that telescopes about an inner body tube (2).

6. I claim the present invention, incorporates a gas seal (4) flare at the distal end of the inner body tube (2) that provides a gas tight seal against the smooth inner bore of the outer body tube (9). The gas seal (4) applies sufficient contact to provide a gas seal while allowing free telescoping movement of the inner body tube (2) and the outer body tube (9) members.

6a. The gas seal (4) is produced by injection molding the thin sealing edge at the distal end of the inner body tube (2) thereby eliminating the need for ‘o’-rings or other add-on seal materials or components.

7. I claim the present invention, incorporates a telescoping action feature and is accomplished by a protruding lug (23) within the outer body tube (9) that resides and is retained within the trough (20) thereby eliminates the possibility of separation of the inner body tube (2) and the outer body tube (9) components.

8. I claim the present invention, position locking or securing is accomplished by multiple twist-lock receivers (3) located and connected perpendicular to the trough (20). The twist-lock receivers (3) have a generous taper or radius entrance (25) allowing a non-specific position alignment of the desired length but within the general area of the lug (23) to the twist-lock receivers (3) entrance.

9. I claim the present invention, position or length indicators of printed, labeled or engraved means can be applied to assist the user in finding the approximate location of the entrance of the twist-lock receivers (3).

10. I claim the present inventions, trough (20) provides a slot and a smooth lug stop/wall (16) for the lug (23) to traverse against during the telescopic movement of the inner body tube (2) and the outer body tube (9) members.

11. I claim the present inventions, lug detent (18) and a lug detent ramp (19) provide the means to engage and secure the lug (23) with a felt click and a firm stop of travel by the lug stop/lock wall (16).

12. I claim the present inventions, eight slots (more or less) about the conical taper (7) of the distal end provide a means of flexing the plastic without damage while extracting the coring pin method used during the injection molding process of the outer body tube (9).

13. I claim the present inventions, cannula head (1) instrument gas sealing assembly can be of various designs thereby not limiting this device to a specific manufacture.
